(19) United States
(12) Patent Application Publication
(10) Pub. No.: US 2015/0202410 A1
(43) Pub. Date: Jul. 23, 2015

(54) APPARATUSES FOR STEERING CATHETERS

(71) Applicant: Cath Med Ltd., Nazareth (IL)

(72) Inventor: Marwan Odeh, Kfar-Yassif (IL)

(73) Assignee: Cath Med Ltd., Nazareth (IL)

(21) Appl. No.: 14/421,854

(22) PCT Filed: Aug. 15, 2013
(86) PCT No.: PCT/IL2013/050694

§ 371 Date: Feb. 16, 2015

Related U.S. Application Data
(60) Provisional application No. 61/683,802, filed on Aug. 16, 2012.

(51) Int. Cl. A61M 25/01; A61B 17/43; A61B 10/02

(52) U.S. Cl. CPC A61M 25/0147; A61B 10/0291; A61B 17/43

(57) ABSTRACT

A steerable catheter device, comprising: a deflector configured with at least one groove, where the groove is a missing section disposed across a part of a cross-section of the deflector; and, a pull wire configured to effectuate bending of the deflector at the groove, being connected on the deflector distally of the groove.

### RELATED APPLICATION/S

This application claims the benefit of priority under 35 USC 119(e) of U.S. Provisional Patent Application No. 61/683,802 filed Aug. 16, 2012, the contents of which are incorporated herein by reference in their entirety.

### FIELD AND BACKGROUND OF THE INVENTION

The present invention, in some embodiments thereof, relates to catheters and, more particularly, but not exclusively, to deflectors for steering catheters.

WO2006020055, the disclosure of which is incorporated herein by reference, describes a cannula, a straightener, and a cannula and straightener combination for insertion into tissue, the cannula including an elongated rigid hollow tube having a proximal end, a distal end, and a passageway extending therebetween. The distal end includes a memory of directionality to bend about a radius.

U.S. Pub. App. No. 20070123750, the disclosure of which is incorporated herein by reference, describes a catheter apparatus including a catheter having an electroactive polymer inside. The electroactive polymer is configured to control either or both shape and/or direction of the catheter in a vessel as the electroactive polymer is electrically activated.

WO2006130435 and U.S. Pub. App. No. 20060270975, the disclosures of which are incorporated herein by reference, describe a steerable catheter having a steering portion incorporating a spear cut junction of catheter material, thereby providing for a gradual change in flexibility in the steering portion.

U.S. Pub. App. No. 20050107678, the disclosure of which is incorporated herein by reference, describes methods of manipulating a catheterization apparatus involving providing a catheter having a flexible shaft with a preformed curve on a distal end of the flexible shaft, one or more electrodes disposed along the distal end of the flexible shaft, and a shape deflection area defined as a region on the flexible shaft between the preformed curve and a generally straight proximal portion of the flexible shaft.

U.S. Pub. App. No. 20040215137, the disclosure of which is incorporated herein by reference, describes a syringe comprising a cylindrical reservoir, closed at its upstream part by a closure element capable of being moved by a driving means, and closed at its downstream part by an injector comprising at least an injection duct and said reservoir being secured to the body of the syringe, said syringe is such that the diaphragm, compatible with the active principle, isolates the latter from the injector and said diaphragm can be passed through by the liquid when pressurized for injection.

Wo200403752, the disclosure of which is incorporated herein by reference, describes a guide catheter with a medical lead wherein the lead may be used as a pull wire to steer the guide catheter.

U.S. Pub. App. No. 20030114833, the disclosure of which is incorporated herein by reference, describes compound steering assemblies, usable in both diagnostic and therapeutic applications, which enable a physician to swiftly and accurately steer the distal section of the catheter in multiple planes or complex curves to position and maintain ablation and/or mapping electrodes in intimate contact with an interior body surface.

U.S. Pub. App. No. 20020016542, the disclosure of which is incorporated herein by reference, describes methods and apparatuses for displaying and using a shaped field of a repositionable magnet to move, guide, and/or steer a magnetic seed or catheter in living tissue for medicinal purposes.

U.S. Pat. No. 6,594,517, the disclosure of which is incorporated herein by reference, describes a method and apparatus for generating a controlled torque of a desired direction and magnitude in an object within a body, particularly in order to steer the object through the body, such as a catheter through a blood vessel in a living body, by producing an external magnetic field of known magnitude and direction within the body, applying to the object a coil assembly including preferably three coils of known orientation with respect to each other, preferably orthogonal to each other, and controlling the electrical current through the coils to cause the coil assembly to generate a resultant magnetic dipole interacting with the external magnetic field to produce a torque of the desired direction and magnitude.

U.S. Pat. No. 5,603,697, the disclosure of which is incorporated herein by reference, describes a medical catheter steering construction that is used for steering ablation catheters that utilize coaxial transmission lines.

### SUMMARY OF THE INVENTION

An aspect of some embodiments of the invention relates to providing a steerable catheter device which is associated with a catheter for assisting the catheter with accessing and/or interfacing with a body lumen. Optionally, the body lumen is not a natural lumen, but one created by pushing through the steerable catheter device through body tissue. In an embodiment of the invention, the steerable catheter device is inserted in the lumen of the catheter. In an embodiment of the invention, the device optionally also includes the catheter or any one of a plurality of interchangeable catheters. In some embodiments of the invention, the device is adapted to be steered using a steering mechanism including a deflector with a groove cut therein, such that bending of the catheter occurs at the groove cut into the deflector. In an embodiment of the invention, the groove is a missing section across a part of a cross-section of the deflector. In an embodiment of the invention, the deflector also functions as a guide wire, pull wire and/or stylet. In some embodiments of the invention, at least a portion of the deflector is elastically and/or plastically deformable. Optionally, the groove is elastically deformed. Optionally, the device is disposable, being discarded after being used to perform a procedure and/or treatment.

In some embodiments of the invention, the deflector is disposed axially and within the catheter substantially along the catheter's length.

In some embodiments of the invention, more than one groove is provided to the deflector. Optionally, each groove is associated with a separate wire. Optionally, a plurality of grooves are provided to the deflector in a coordinated fashion, for example having two deflecting sections with 90 degrees rotation to each other.

In some embodiments of the invention, an over-layer is provided around the deflector. Optionally, the over-layer is provided to the deflector only where the groove, or grooves if more than one, is cut into the deflector. The over-layer is optionally pre-weakened at a particular point in order to control where the bending occurs. The over-layer is optionally used to distribute the bending forces along its length to protect the deflector, and to protect the internal surface of the catheter from any contact with the bending section of the deflector. In some embodiments of the invention, the over-layer prevents or resists collapsing into the groove lumen of the deflector when being used with a catheter.

In an embodiment of the invention, the deflector is sized for being used interchangeably with a plurality of catheters, for example catheters designed for use with and/or for performing different procedures. Exemplary procedures include chorionic villus sampling (“CVS”), in-vitro fertilization (“IVF”) and intrauterine insemination (“IUI”). In an embodiment of the invention, the interchangeable catheters are configured to conform to the deflector and/or to have a working channel therein where the deflector is insertable. In an embodiment of the invention, the deflector is adjustable in size (e.g. overall length), for example by cutting at least some length off of the distal end of the deflector, in order to move a pre-cut groove further to the end of the deflector. In some embodiments of the invention, the length is adjusted by repositioning the deflector at the handle, for example by spooling more deflector in a distal direction or withdrawing some of the deflector in a proximal direction.

An aspect of some embodiments of the invention relates to a method of steering a catheter using a deflector/guide wire during a CVS procedure comprising bending the deflector after it has been inserted into the patient's body. In some embodiments of the invention, the deflector is removed after the catheter has arrived at a target area, for example inside or near the chorionic villus.

An aspect of some embodiments of the invention relates to a method of steering a catheter using a deflector with a groove cut therein. In some embodiments of the invention, steering is performed by at least one of rotating the catheter about its longitudinal axis and activating the deflector to adjust the angle of the distal end of the catheter. In an embodiment of the invention, the deflector is activated by moving a pull wire in a proximal direction. In an embodiment of the invention, the deflector is straightened by moving the pull wire in a distal direction. In an embodiment of the invention, the deflector is activated by sliding at portion of the deflector relative to the catheter. Optionally, the catheter functions as a pull wire.

There is provided in an exemplary embodiment of the invention, a steerable catheter device, comprising: a deflector configured with at least one groove, where the groove is a missing section disposed across a part of a cross-section of the deflector; and, a pull wire configured to effectuate bending of the deflector at the groove, said pull wire being connected on the deflector distally of the groove.

In an embodiment of the invention, the steerable catheter device further comprises a handle connected to a proximal end of the deflector, where the handle is removably connected to a catheter.

In an embodiment of the invention, the steerable catheter device further comprises a catheter, wherein the deflector is detachably located within a lumen of the catheter such that bending of the deflector by the pull wire also causes bending of the catheter.

In an embodiment of the invention, the steerable catheter device further comprises a handle with a lever located on the handle and connected to the pull wire, wherein movement of the lever effectuates movement of the pull wire and bending of the deflector. In an embodiment of the invention, the lever is provided with a plurality of removably lockable positions. In an embodiment of the invention, the plurality of removably lockable positions correlate to known degree increments of bending of the deflector.

In an embodiment of the invention, the pull wire is connected to the deflector by at least one of laser welding and an over-wrap.

In an embodiment of the invention, the steerable catheter device further comprises an over-layer around the deflector and pull wire. In an embodiment of the invention, the over-layer is heat shrunk on the deflector. In an embodiment of the invention, the over-layer retains the pull wire close to the deflector during bending.

In an embodiment of the invention, the steerable catheter device includes a plurality of grooves. In an embodiment of the invention, the plurality of grooves each corresponds to a single pull wire for individual control of bending of each groove.

In an embodiment of the invention, the steerable catheter device further comprises a catheter with multiple lumens where the deflector is detachably located in one of the lumens.

In an embodiment of the invention, the deflector and the pull wire together have a substantially circular cross-section.

In an embodiment of the invention, the deflector is plastically deformable.

In an embodiment of the invention, the groove is elastically deformable.

In an embodiment of the invention, the steerable catheter device further comprises a sharpened tip located at the distal end of the catheter for retrieving samples.

In an embodiment of the invention, the groove of the deflector is bendable up to 90 degrees from the longitudinal axis of the deflector.

There is further provided in an exemplary embodiment of the invention, a steerable catheter device kit, comprising: a plurality of catheters; and, a deflector configured to be compatible with any of the plurality of catheters.

In an embodiment of the invention, at least one of the plurality of catheters is configured for performing a CVS, IVF or IUI procedure.

There is further provided in an exemplary embodiment of the invention, a method of using a steerable catheter device, comprising: inserting the steerable catheter device including a deflector and a catheter; navigating the steerable catheter device to a target area, wherein navigating includes at least one of deflecting the deflector at a groove located thereon; and, removing at least a portion of the steerable catheter device.

In an embodiment of the invention, the method of using a steerable catheter device further comprises using medical imaging for at least one of the inserting, navigating and removing.

In an embodiment of the invention, removing includes removing the deflector but leaving the catheter in situ. In an embodiment of the invention, the method of using a steerable catheter device further comprises attaching a syringe to the catheter for retrieving a sample from the target area. In an embodiment of the invention, the method of using a steerable catheter device further comprises depositing at least one of semen and fertilized eggs at the target area. In an embodiment of the invention, the method of using a steerable catheter device further comprises using a sharpened tip to retrieve a sample from the target area.

In an embodiment of the invention, the target area is in the chorionic villus.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example, are not necessarily to scale, and are for purposes of illustrative discussion of embodiments of the invention. In this regard, the description taken with the drawings makes apparent to those skilled in the art how embodiments of the invention may be practiced.

In the drawings:

FIG. 1 is a perspective view of a distal end of a steerable catheter, in accordance with an exemplary embodiment of the invention;

FIG. 2 is a cross-sectional view of the distal segment of the steerable catheter shown in FIG. 1, in accordance with an exemplary embodiment of the invention;

FIG. 3 is a top or bottom view looking along the longitudinal axis of the catheter shown in FIG. 1, in accordance with an exemplary embodiment of the invention;

FIGS. 4 and 5 show the distal end of the steerable catheter shown in FIG. 1 activated in opposing directions, in accordance with an exemplary embodiment of the invention;

FIG. 6 is a perspective view of an alternative steerable catheter apparatus, in accordance with an exemplary embodiment of the invention;

FIG. 7 is a perspective view of an alternative steerable catheter apparatus, in accordance with an exemplary embodiment of the invention;

FIGS. 8A and 8B are perspective views showing a deflector with multiple grooves, in accordance with an exemplary embodiment of the invention;

FIG. 9 is a perspective, partially exploded view of a steerable catheter apparatus including the handle, in accordance with an exemplary embodiment of the invention;

FIG. 10 is a perspective, partially exploded view of the deflector/guide wire assembly and handle, in accordance with an exemplary embodiment of the invention;

FIG. 11 is a side view of the deflector and linear guide of the handle, in accordance with an exemplary embodiment of the invention;

FIG. 12 is a side view of the groove portion of the deflector/guide wire, in accordance with an exemplary embodiment of the invention;

FIG. 13 is a side, cross sectional view of a portion of the handle showing selectable positions, in accordance with an exemplary embodiment of the invention;

FIG. 14 is a perspective view showing deflection of the groove portion of the deflector/guide wire, in accordance with an exemplary embodiment of the invention;

FIG. 15 is a flowchart illustrating a method of using a steerable catheter apparatus, in accordance with an exemplary embodiment of the invention;

FIG. 16 is a flowchart illustrating a method of using a steerable catheter apparatus to perform CVS, in accordance with an exemplary embodiment of the invention; and,

FIG. 17 is a flowchart illustrating a method of using a steerable catheter apparatus to perform IVF or IUI, in accordance with an exemplary embodiment of the invention.

### DESCRIPTION OF SPECIFIC EMBODIMENTS OF THE INVENTION

The present invention, in some embodiments thereof, relates to catheters and, more particularly, but not exclusively, to deflectors for steering catheters.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

In an embodiment of the invention, a catheter deflector and/or guide wire is provided which is used to angle a catheter during navigation of a body lumen. In some embodiments of the invention, the deflector is also used as a guide wire and/or pull wire and the same structure accomplishes both deflecting and/or guiding and/or pulling. In some embodiments, the deflector is usable with a plurality of interchangeable catheters. In some embodiments of the invention, the same deflector is used to insert a catheter, withdraw, then insert a different catheter, in the course of the same procedure or treatment of a patient.

In an embodiment of the invention, a catheter is provided which, for example, evacuates sampled tissue, and delivers fluids, chemical compounds, tissues, implants and/or surgical equipment to or from a surgical site disposed within a body or body lumen of a patient. The “distal” end of the catheter is the end which is first to be introduced into the patient's body or body lumen. The “proximal” end of the catheter is the end of the catheter opposite the distal end, or the end closest to the attending medical professional. Distal and proximal as they are used herein with respect to other structures or directions of motion mirror this convention. The catheter, in some embodiments of the invention, has a tubular body the distal end of which is deflected by the deflector to assist with steering the catheter.

Activating the deflection of the distal end is accomplished, in an embodiment of the invention, by sliding at least a portion of the deflector in relation to the catheter, taking advantage of a width, W, of a groove cut into the deflector to allow for deflection. Activating the deflection of the distal end is accomplished, in an embodiment of the invention, by pulling or pushing the deflector causing bending of the deflector at the groove, which also causes bending of the catheter located on the deflector.

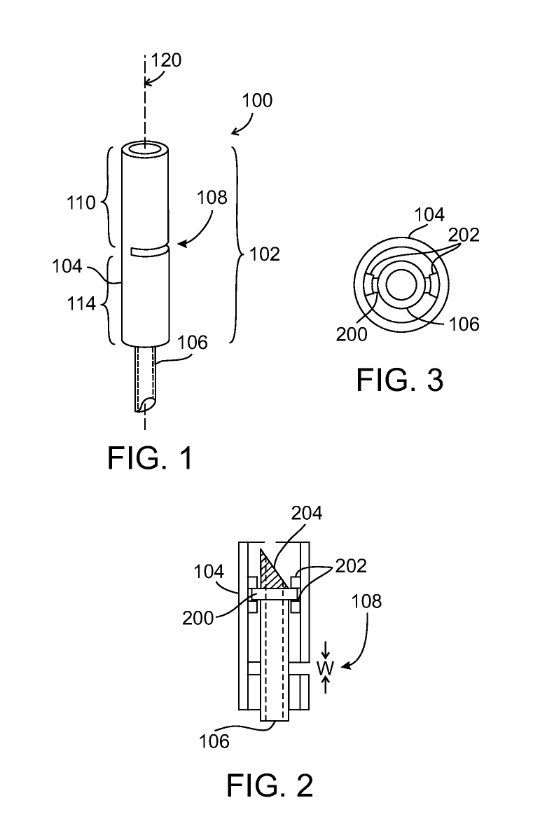

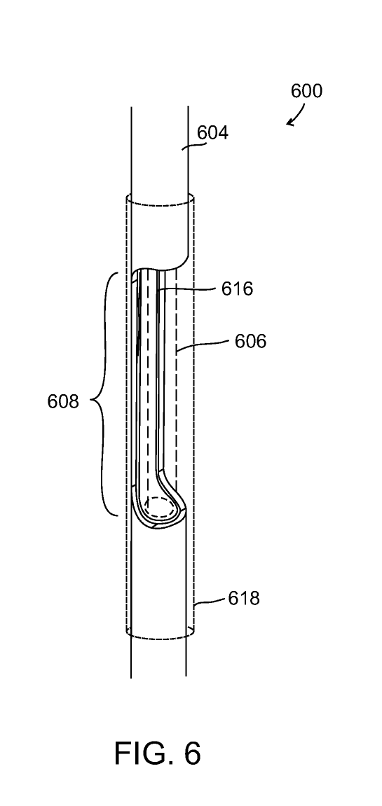

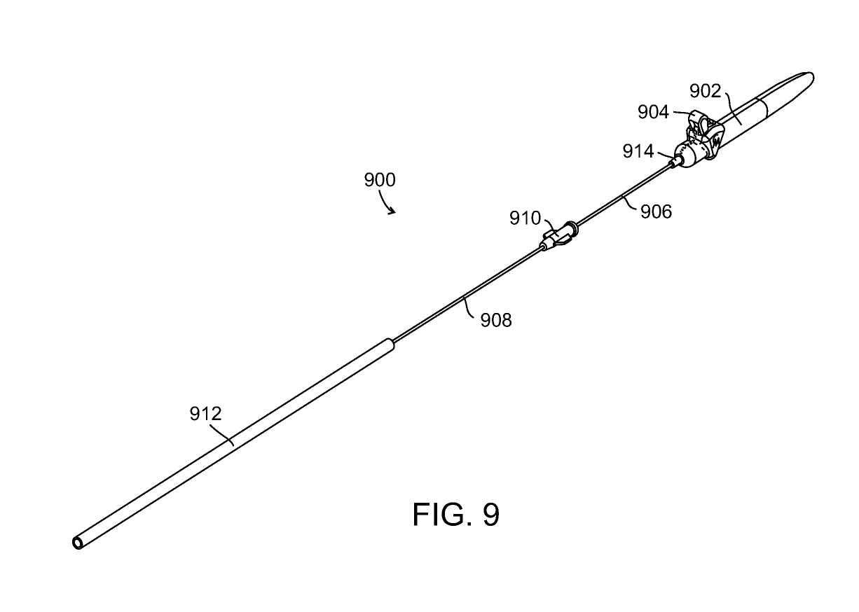

It should be understood that there are a plurality of embodiments conceived by the inventor for providing a deflector which steers a catheter in a body lumen for the purposes of navigating the catheter through the lumen to a target area in the patient. For example, FIGS. 1-5 describe a first embodiment. FIGS. 6, 7 and 8A-8B describe additional embodiments, and FIGS. 9-14 describe yet another embodiment.

Referring now to the drawings, FIG. 1 is a perspective view of a distal end 102 of a steerable catheter apparatus 100, in accordance with an exemplary embodiment of the invention. In an embodiment of the invention, the steerable catheter apparatus 100 comprises a deflector 104 located at the distal end 102, and a catheter 106 which extends substantially the full length of the apparatus 100 and passes through the deflector 104 at the distal end 102. In some embodiments of the invention, the steerable catheter apparatus 100 is used with a deflector/guide wire. In some embodiments of the invention, the deflector 104 and the catheter 106 are cylindrical. The catheter 106 is sufficiently flexible to bend while being advanced and/or retracted through curved and/or or convoluted body lumens. At least one groove 108 is cut into the deflector 104 such that the upper portion 110 of deflector 104 can rotate at least partially around a pivot point 112 with respect to a lower portion 114 of deflector 104 (upper portion 110 and lower portion 114 separated by groove 108) thereby deflecting the distal end 102 with respect to a longitudinal axis 120 of the steerable catheter apparatus 100, in an embodiment of the invention. Thus, when the deflector 104 rotates, while the catheter is inserted therein, the catheter also deflects.

In an embodiment of the invention, catheter 106 is separate from the deflector 104, and is removably inserted into the deflector 104 at the time the procedure is performed. Optionally, the deflector 104 is removed from the catheter 106 during the procedure.

In an embodiment of the invention, the deflector 104 and the catheter 106 are made from the same flexible and/or elastically deformable material, for example polytetrafluoroethylene (PTFE). The flexibility and/or deformation of the material enables the two portions 110, 112 of the deflector to rotate with respect to each other around the pivot point 112. In some embodiments of the invention, the deflector 104 and/or catheter 106 are constructed of any biocompatible material, such as medical grade plastics or metals, like stainless steel.

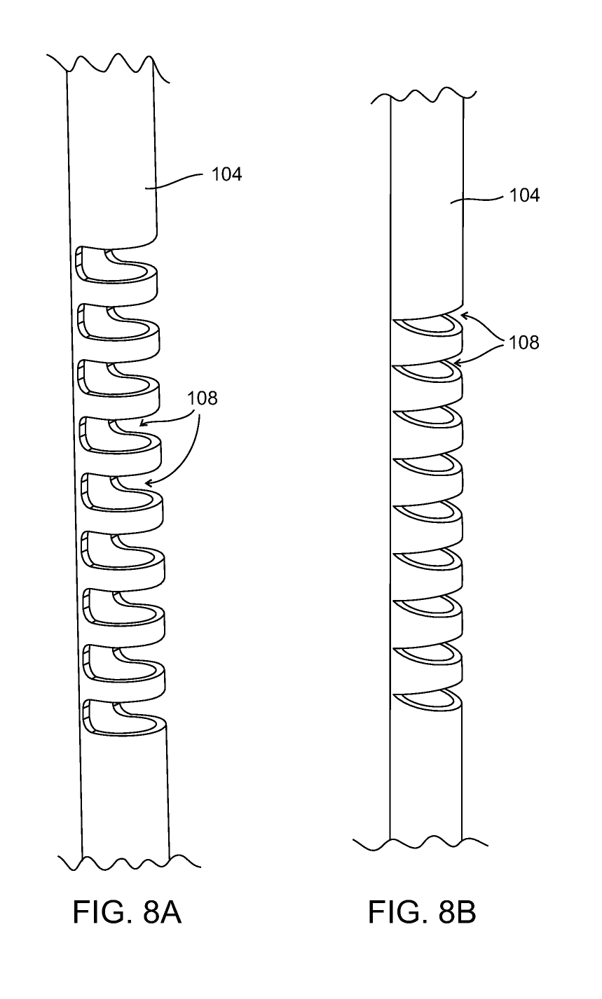

FIG. 2 is a cross-sectional view of the distal segment of the steerable catheter shown in FIG. 1, in accordance with an exemplary embodiment of the invention. In an embodiment of the invention, the catheter 106 is removably or permanently attached to the deflector 104 by providing a ledge 200 to the distal end of the catheter 106 which is positioned between two bulges 202 provided to the deflector 104. In an embodiment of the invention, the deflector 104 is attached to the catheter 106 at a location on the deflector 104 and catheter 106 which is distal of the groove 108 (or last distal groove if there is more than one groove, for example as shown in FIGS. 8A-8B), or in the upper portion 110. Optionally, the catheter 106 is attached to the deflector 104 by an adhesive with or without the use of ledge(s) 200 and counterpart bulges 202, for example if the catheter 106 is snugly fit within the deflector 104, but where a snug fit is not so snug that it still allows siding between the deflector 104 and the catheter 106.

In an embodiment of the invention, the maximal deflection achievable by the deflector 104 and/or the catheter 106 may be limited at least partly by the length of the upper portion 110, the width, W, of the groove 108 and the ratio between the inner radius of the deflector 104 divided by the radius of the catheter 106, which is indicative to the dimensions of the clearances present between the catheter 106 and the enclosing deflector 104. The lateral clearances present between the inner surface of the deflector 104 and the outer surface of the catheter 106, may limit the angle of deflection while the catheter 106 is being axially stressed, so in an embodiment of the invention, by suitably selecting the dimensions of the catheter 106 and the lumen size of the deflector 104, bending of the deflector that might restrict or block the axial motion of the catheter 106 relative to the deflector 104 is at least partially avoided.

In an embodiment of the invention, the overall diameter of the steerable catheter apparatus is approximately 1 mm. In an embodiment of the invention, the diameter of the catheter 106 is approximately 0.25 mm. In some embodiments of the invention, the internal diameter of the deflector 104 is approximately 0.7 mm. In some embodiments of the invention, the width of the groove, W, is 3.5 mm in some embodiments of the invention, the maximal angles of deflection are ±45°. In some embodiments of the invention, the maximal angles of deflection are ±90°. In some embodiments of the invention, the maximal angles of deflection are greater than 90°.

In an embodiment of the invention, an optional sharpened tip 204 provides for penetrating a tissue at the targeted site within the body of a patient. Such sharpened tip 204 is typically made of a stiff material such as metal or a compliant stiff plastic resin. In some embodiments of the invention, activation and/or depth of insertion and/or duration of sampling and/or volume of sampling, as examples, of the tip 204 are controlled automatically, for example by a controller/computer.

FIG. 3 is a top or bottom view looking along the longitudinal axis 120 of the steerable catheter apparatus 100 shown in FIG. 1, in accordance with an exemplary embodiment of the invention. In an embodiment of the invention, FIG. 3 shows how the catheter 106 can be removed from the deflector 104 by rotating one or both of them to unlock the ledge 200 from the bulges 202.

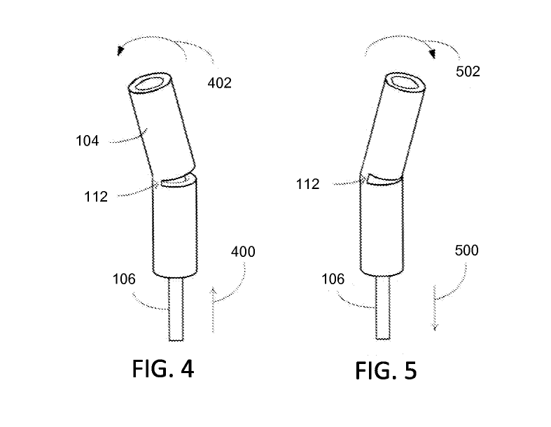

FIGS. 4 and 5 show the distal end 102 of the steerable catheter apparatus 100 shown in FIG. 1 being deflected in opposing directions 402, 502, in accordance with an exemplary embodiment of the invention. In an embodiment of the invention, the catheter 106 acts as a pull wire to activate deflection of the deflector 104. As described above, the catheter 106 is slidingly movable along the longitudinal axis 120 with respect to the deflector 104 except for the distal end of the catheter 106, which is either detachably or permanently attached to the upper portion 110 of the deflector 104. Upper portion 110 of the deflector 104, and also the corresponding portion of the catheter 106 located therein, is deflected by distally pushing 400 (shown in FIG. 4) or proximally pulling 500 (shown in FIG. 5) the catheter 106 relative to the body of the deflector 104, in an embodiment of the invention. Distally pushing 400 the catheter 106 brings about deflecting in the direction of curved arrow 402 and around pivot point 112. Proximally pulling 500 the catheter 106 causes deflection towards the direction shown by curved arrow 502. The upper limit of the range of angles by which the upper portion 110 is deflected in the directions of arrow 402 and/or arrow 502 is at least partially defined by the width, W, of groove 108 and/or the inner radius (from the longitudinal axis 120) of deflector 104 and external radius of catheter 106, referred to elsewhere herein as “clearance”.

FIG. 6 is a perspective view of an alternative steerable catheter apparatus 600 with a groove 608 cut into the deflector 604, in accordance with an exemplary embodiment of the invention. In an embodiment of the invention, the deflector comprises at least two layers including an outer layer and an inner, reinforcing layer 616. In an embodiment of the invention, the outer layer is to shield the inner surface of the body lumen. In an embodiment of the invention, the inner layer structurally reinforces the outer layer particular in the bendable section located at the groove 608. In some embodiments of the invention, the inner, reinforcing layer 616 is provided to the deflector 604 to prevent failure of the deflector 104 during deflection, where it is cut at the wide groove 108. In some embodiments of the invention, the deflector 604 extends the length of the catheter 606, or substantially so. Optionally, one or both layers are metal, for example the inner, reinforcing layer 616 can be metal. Optionally, one or both layers are plastic, for example the outer layer can be plastic.

In some embodiments of the invention, an over-layer 618 is provided to the deflector 604 at least to provide additional structural reinforcement to the deflector 604 where the wide groove 608 is cut therein. Optionally, the over-layer 618 is pre-weakened in order to select where along the longitudinal axial length of the groove 608 deflection occurs. Additionally, alternatively and/or optionally, the over-layer distributes the forces experienced during deflection along its length to reduce stress on a particular part of the deflector 604. In some embodiments of the invention, the over-layer 618 provides lateral stability to the steerable catheter apparatus 600, thereby resisting forces which would collapse the lumen of the apparatus 600 through which the catheter 606 passes. Optionally, the over-layer 618 is located on the external surface of the deflector 604 where the wide groove 608 is cut.

In an embodiment of the invention, deflection of the steerable catheter apparatus 600 is achieved by moving the catheter 606 with respect to the deflector 604, such as is described in other embodiments herein.

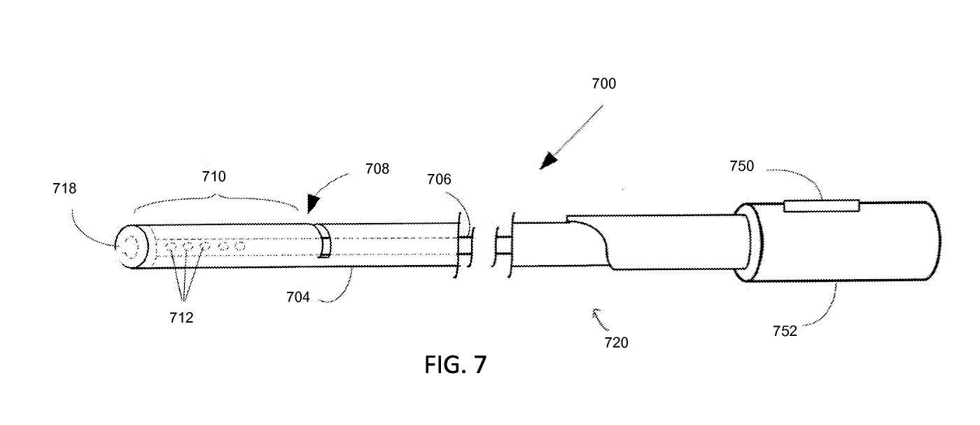

FIG. 7 is a perspective view of another steerable catheter apparatus 700, in accordance with an exemplary embodiment of the invention. Apparatus 700 has a deflector 704 in which circumferential groove 708 defines an upper portion 710. Catheter 706, the distal end of which is secured to the distal end 718 of the deflector 704, is housed within deflector 704 such that it can be moved axially within deflector 704 in order to activate deflection of the steerable catheter apparatus 700.

A lever 750 is installed on gripping handle 752 that is attached at the proximal end 720 of deflector 704. In an embodiment of the invention, lever 750 is configured to proximally and/or distally move the catheter 706, which is operatively connected to the lever 750, thereby deflecting upper portion 710 of the steerable catheter apparatus 700.

In some embodiments of the invention, at least one aperture 712 is provided to the catheter 706 for inducing negative pressure within the lumen of the deflector 704. In such an embodiment, the catheter 706 is connected to a vacuum source.

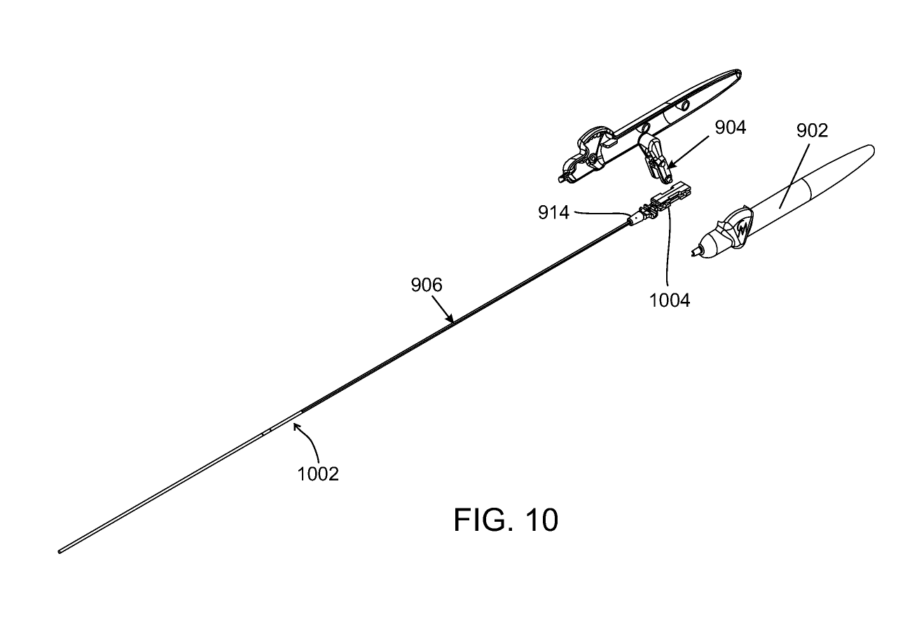

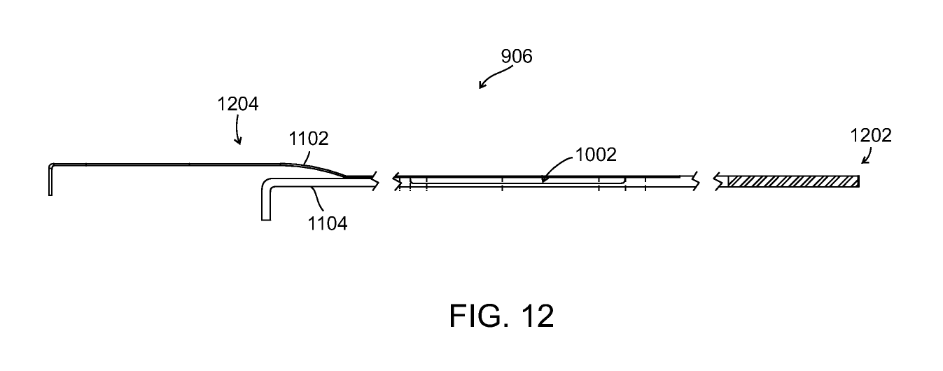

FIG. 9 is a perspective, partially exploded view of a steerable catheter apparatus 900 including a handle 902, a deflector/guide wire 906 and a catheter 908, in accordance with an exemplary embodiment of the invention. It should be understood that the handle portion, along with lever 904 and deflector/guide wire 906 are usable with any number of interchangeable catheters, for example, catheters which are intended to be used to perform CVS, IVF and IUI procedures. Catheter 908 is placed over the deflector/guide wire 906, in an embodiment of the invention. Deflector/guide wire 906 is shown and described in more detail with respect to FIGS. 10, 11, 12 and 14. In an embodiment of the invention, lever 904 is used to move deflector 906 in a proximal direction or distal direction in order to cause deflection of the catheter 908 or straighten the catheter 908, respectively, as described in more detail with respect to FIG. 12 and elsewhere herein.

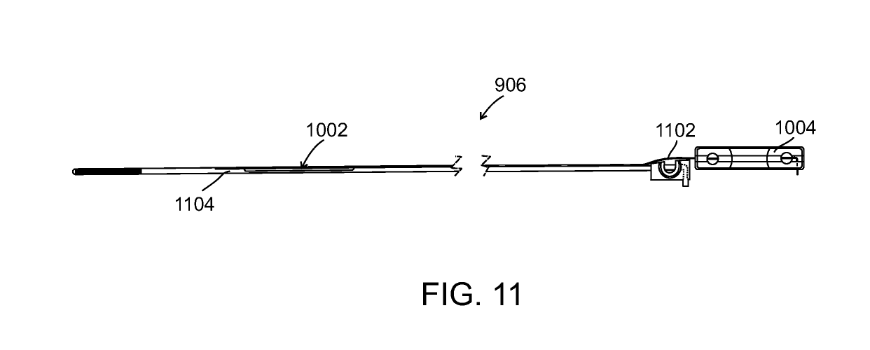

The catheter 908 is secured to the handle 902 using a Luer fitting 910, in some embodiments of the invention. In some embodiments of the invention, the Luer fitting 910 is removably attached to a cap 914, the cap 914 being used in some embodiments of the invention to operatively secure the deflector to the linear guide 1004 (shown and described in more detail with respect to FIG. 10) optionally via an guide connection (seen in FIG. 11). It is conceived that in the course of a procedure, the deflector 906 may need to be removed from the lumen of the catheter 908; in an embodiment of the invention, the cap 914 is configured to easily disengage from the Luer fitting 910 in order to remove the deflector 906 while still leaving the catheter 908 in situ. In some embodiments of the invention, the cap 914 fits within the Luer fitting 910 using a compression fit. In some embodiments of the invention, the cap is provided with ribs on its exterior which are optionally configured to act as counterparts to female slots on the interior of the Luer fitting 910. Optionally, the ribs on the exterior of the cap 914 are also used as gripping points for the hand of the attending medical professional during removal of the deflector 906 from the catheter 908.

In some embodiments of the invention, an over-layer (not shown) is provided around the deflector 906. Optionally, the over-layer is provided to the deflector 906 where the groove 1002 (shown in more detail in FIG. 10), or grooves if more than one, is located on the deflector 906. The over-layer is optionally used to distribute at least some of the bending forces along its length to protect the deflector 906 and/or to protect the internal surface of the catheter 908 from any contact with the external surface of the bending section of the deflector 906. In some embodiments of the invention, the over-layer prevents or resists forces collapsing the groove 1002 lumen of the deflector 906 when being used with a catheter 908. In an embodiment of the invention, the over-layer is heat shrunk onto the deflector 906 and/or pull wire 1102 (described in more detail below). In an embodiment of the invention, the over-layer holds the pull wire 1102 in close proximity to the deflector 906 when the pull wire 1102 is pulled to cause bending and/or deflection.

In an embodiment of the invention, the cap 914 secures the proximal end of the over-layer to the deflector 906.

A protective tube 912 is also shown in FIG. 9, which is optionally used to protect the steerable catheter apparatus 900 during shipping and/or storage in embodiments where the deflector 906 is pre-inserted into the catheter 908 and/or pre-mounted on handle 902 during manufacture prior to shipping and use. In an embodiment of the invention, the protective tube 912 is removed from the catheter 908 prior to use.

FIG. 10 is a perspective, partially exploded view of the deflector/guide wire 906 assembly and handle 902, in accordance with an exemplary embodiment of the invention. The deflector 906 is operatively connected to a linear guide 1004 which is operatively connected to the lever 904, in an embodiment of the invention. Through this arrangement, movement of the lever 904 translates to movement of the deflector 906. A groove 1002 in the deflector 906 is shown in FIG. 10 as an example of where and/or how the groove 1002 could be implemented, in an embodiment of the invention. In an embodiment of the invention, the groove 1002 is any pre-weakened section of the deflector 906, and may not necessarily take the exemplary structure pictured in the Figures. Optionally, the groove 1002 is not axially symmetric. In some embodiments of the invention, at least a portion of the deflector is elastically and/or plastically deformable. Optionally, the groove is elastically deformed.

FIG. 11 is a side view of the deflector 906 and linear guide 1004 of the handle 902, in accordance with an exemplary embodiment of the invention. In an embodiment of the invention, the deflector 906 comprises a pull wire 1102 and a stylet 1104, which are attached together. Groove 1002 is shown in stylet 1104 where it is conceived that movement of the pull wire 1102 by the linear guide 1004 (which is in turn connected to the lever 904) causes the deflector 906 to bend or deflect at the groove 1002 location.

FIG. 12 is a side view of the of the deflector/guide wire 906 including the groove 1002 portion, in accordance with an exemplary embodiment of the invention. In an embodiment of the invention, the pull wire 1102 extends distally past the groove 1002 in order to effectuate deflection when the pull wire 1102 is moved in a proximal direction. In FIG. 12, the distal end 1202 is inserted into the patient first. The proximal end 1204 is where a handle would be located and where the pull wire 1102 and stylet 1104 would be mounted. In an embodiment of the invention, the pull wire 1102 is approximately 267 mm in length. In an embodiment of the invention the stylet 1104 is approximately 260 mm in length. In some embodiments of the invention, the device is approximately 290 mm in length. In some embodiments of the invention, the groove 1002 is located approximately 30 mm from the distal end of the deflector/guide wire 906. In some embodiments of the invention, the groove 1002 is approximately 20 mm long. It should be understood that these are exemplary dimensions for an embodiment only, and that depending on the intended use and manufacturing deviations the dimensions can change without substantially affecting the performance of the device. In an embodiment of the invention, the pull wire 1102 is configured as a thin strip. In some embodiments of the invention, the pull wire 1102 is integrally formed with stylet 1104. In some embodiments of the invention, the pull wire 1102 is not integrally formed with the stylet 1104. Optionally, the pull wire 1102 is laser welded onto the stylet 1104. Optionally, the over-layer secures the pull wire 1102 to the stylet 1104. In an embodiment of the invention, the deflector and the pull wire together have a substantially circular cross-section.

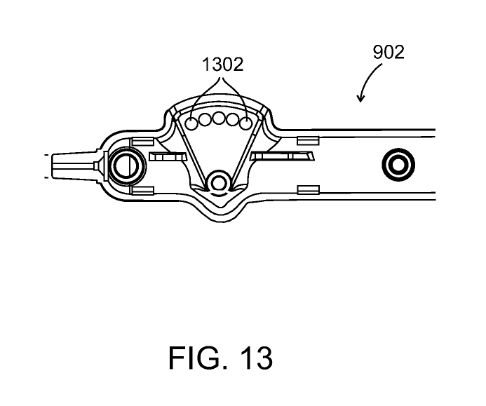

FIG. 13 is a side, cross sectional view of a portion of the handle 902 showing selectable positions 1302 for the lever 904, in accordance with an exemplary embodiment of the invention. In an embodiment of the invention, the attending medical professional uses his/her thumb to operate the lever 904 while performing the procedure. When in the most forward or distal position, the deflector 906 is straight, however as the lever is adjusted backwards or proximally, the pull wire 1102 moves proximally, causing the distal end of the deflector 906 to deflect around the groove 1002. In an embodiment of the invention, each selectable position 1302 represents a specific amount (in terms of degrees) of deflection. Each selectable position 1302 acts as a counterpart to a projection on the lever 904 which is designed to removably lock into the selectable positions 1302. While there are five selectable positions 1302 shown, any number can be provided, or even none, in an embodiment where the deflection control is slidable and without removably lockable positioning. In some embodiments of the invention, the selectable positions 1302 are not evenly spaced. In the course of a typical procedure, the lever 904 would be used to deflect and straighten the deflector 906, and therefore the catheter 908, repeatedly as the medical professional navigates the body lumen.

It should be understood that in some embodiments, a lever is not used but some other form of ergonomically acceptable control.

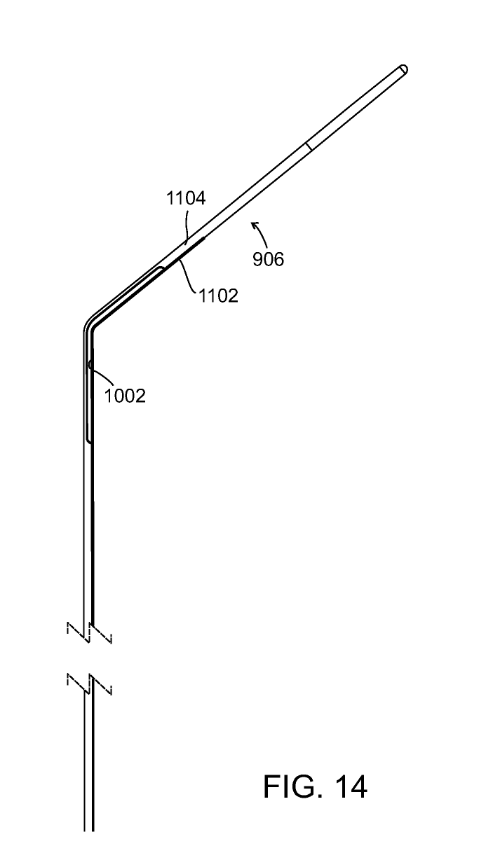

FIG. 14 is a perspective view showing deflection of the groove 1002 portion of the deflector/guide wire 906, in accordance with an exemplary embodiment of the invention. In an embodiment of the invention, the pull wire 1102 of the deflector 906 has been moved in a proximal direction 1402 by the lever 904. The pull wire 1102 has caused the stylet 1104 to bend at the groove 1002, which is weaker than the non-grooved portion of the stylet 1104. In an embodiment of the invention, the deflector 906 bends up to 45 degrees. In an embodiment of the invention, the deflector 906 bends up to 90 degrees. In an embodiment of the invention, the deflector 906 bends up to 120 degrees. In an embodiment of the invention, the deflector 906 bends more than 120 degrees.

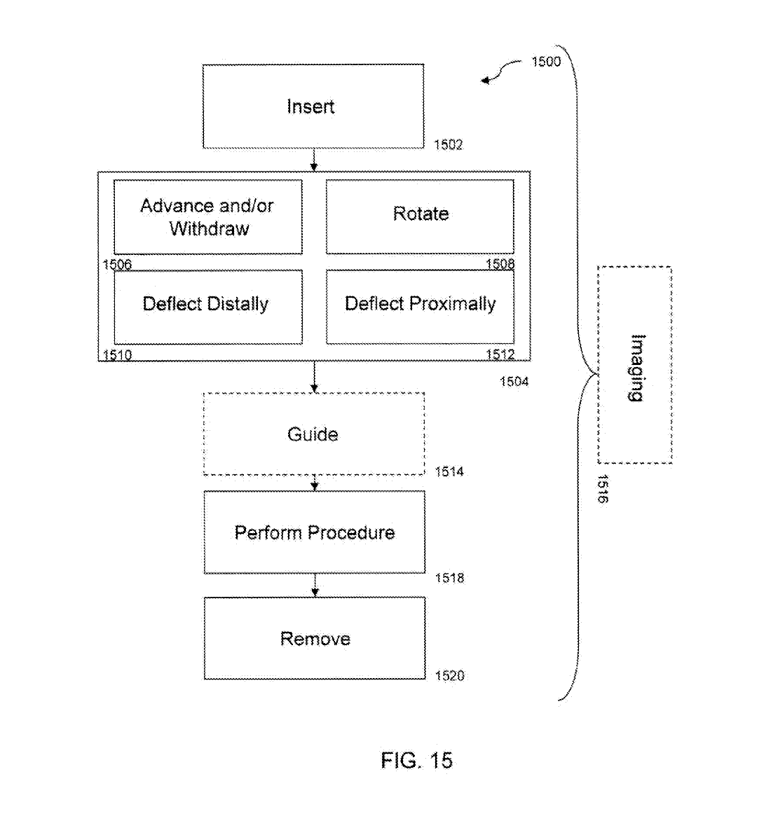

FIG. 15 is a flowchart 1500 illustrating a method of using a steerable catheter apparatus, in accordance with an exemplary embodiment of the invention. In an embodiment of the invention, the steerable catheter apparatus is steered within a body lumen to place the distal end of the steerable catheter apparatus in close proximity to a target area within the patient's body.

Steerable catheter apparatus is inserted (1502) into the patient and is advanced within the body lumen towards the target area, in an embodiment of the invention. In an embodiment of the invention, steerable catheter apparatus is navigated (1504) through and/or around various curves of the body lumen during transit to the target area.

In an embodiment of the invention, navigating (1504) may include one or more of advancing or withdrawing (1506) the steerable catheter apparatus, rotating (1508) the steerable catheter apparatus about its longitudinal axis, deflecting the upper portion distally (1510) or deflecting the upper portion proximally (1512). In an embodiment of the invention, rotating (1508) the steerable catheter apparatus about its longitudinal axis provides for azimuthally rotating the plane containing the respective angles of deflection, thereby pointing the distal end of the catheter towards the desired azimuth angle.

Optionally, a guide wire is used to guide (1514) the steerable catheter apparatus through the body lumen.

In an embodiment of the invention, medical imaging (1516) is optionally used to verify positioning. In some embodiments of the invention, medical imaging (1516) is used prior to the commencement of the procedure to determine the internal disposition of at least a portion of the patient's body. Examples of imaging include camera and/or x-ray and/or ultrasound and/or magnetic resonance. In some embodiments of the invention, the steerable catheter apparatuses described herein are configured to be usable with these various forms of medical imaging, for example plastic components include radiopaque inserts.

In an embodiment of the invention, upon attainment of desired positioning within the patient proximal to the target area, a procedure is performed (1518) on and/or in the target area.

The steerable catheter apparatus is removed (1520) from the patient upon the conclusion of the performing (1518) the medical procedure, in an embodiment of the invention. Optionally, verification that the procedure was completed successfully is conducted prior to removal (1520) of the steerable catheter apparatus from the patient.

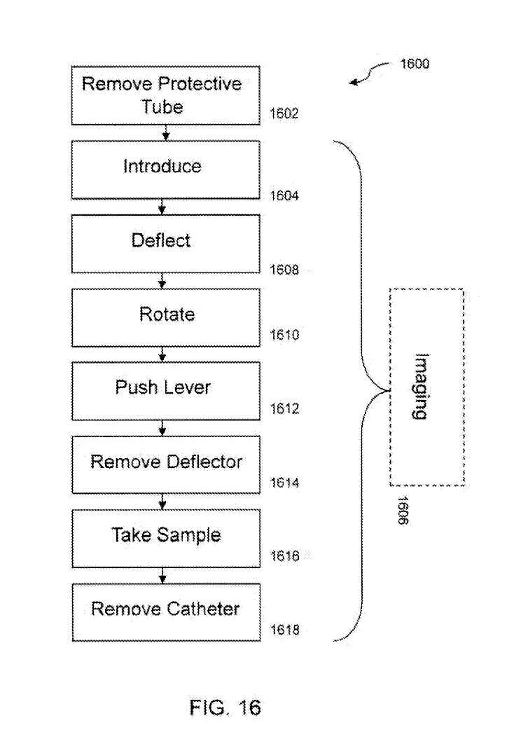

FIG. 16 is a flowchart 1600 illustrating a method of using a steerable catheter apparatus to perform CVS, in accordance with an exemplary embodiment of the invention. CVS is a common minimally invasive procedure in which a tissue sample is removed off of a placenta for further examination and is normally carried out at early stages of pregnancy. Removing (1602) the protective tubing from the catheter is accomplished prior to the steerable catheter apparatus being introduced (1604) and/or advanced through the cervix. The operator optionally monitors (1606) at least the introducing (1604) by means of ultrasonic imaging. The upper portion of the steerable catheter apparatus is deflected (1608) towards an opening in the uterus by suitably rotating (1610) the steerable catheter apparatus about its longitudinal axis while pushing (1612) a lever on a handle proximally or distally as required. Optionally, the deflector is removed (1614) off the catheter when the distal end of the catheter reaches the sample extraction point within the placenta, leaving the catheter in situ at the target area. Taking (1616) a sample tissue off the chorionic villi is accomplished by means of a syringe attached to the proximal end of the catheter after withdrawing the deflector/guide-wire and handle out of the catheter, in an embodiment of the invention. Optionally, negative pressure is introduced into the catheter. Optionally, the distal end of the catheter is moved back and forth to enable better evacuating of a sample of the placenta. Once a sample has been taken (1616) whatever remains of the catheter apparatus in the patient is removed (1618).

In an embodiment of the invention, introducing, deflection and/or rotation (1604, 1608, 1610) of the steerable catheter apparatus in the body lumen exerts no more than 41 grams of force. In an embodiment of the invention, 41 grams of force is adequate to penetrate the Chorionic villi tissue and extract a sample, but it not too forceful so as to damage the placenta and/or the embryonic sac.

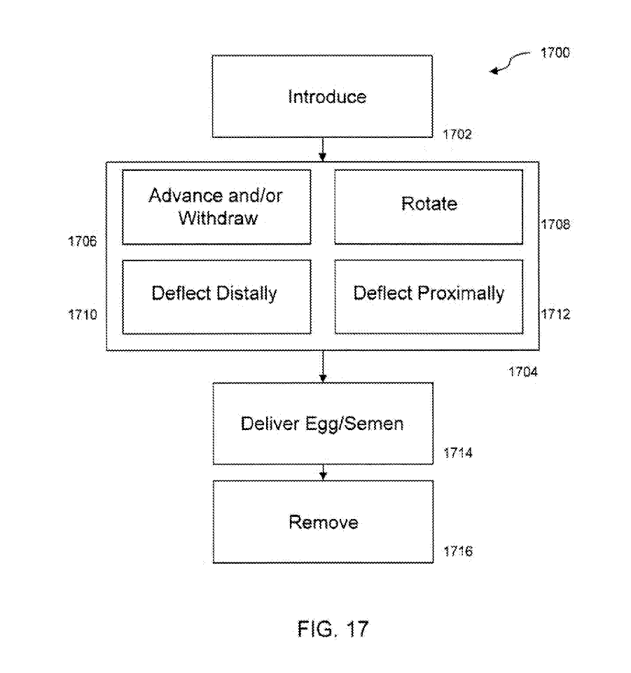

FIG. 17 is a flowchart 1700 illustrating a method of using a steerable catheter apparatus to perform IVF or intrauterine insemination IUI, in accordance with an exemplary embodiment of the invention. In an embodiment of the invention, the steerable catheter apparatus is introduced (1702) through the cervix and into the uterus. The steerable catheter apparatus is navigated (1704) to a target area (for example, where the semen or eggs are to be deposited, depending on which procedure is being performed). Exemplary target areas include, but are not limited to, the openings of the Fallopian tubes and the uterus.

In an embodiment of the invention, navigating (1704) may include one or more of advancing or withdrawing (1706) the steerable catheter apparatus, rotating (1708) the steerable catheter apparatus about its longitudinal axis, deflecting the upper portion distally (1710) or deflecting the upper portion proximally (1712). In an embodiment of the invention, rotating (1708) the steerable catheter apparatus about its longitudinal axis provides for azimuthally rotating the plane containing the respective angles of deflection, thereby pointing the distal end of the catheter towards the desired azimuth angle.

Once the distal end of the steerable catheter apparatus has been navigated (1704) to the target area, semen and/or eggs are delivered (1714) to the target.

The steerable catheter apparatus is removed (1716) from the patient after delivery (1714) at the target area.

In some embodiments of the invention, an attending medical professional knows how far to advance the steerable catheter apparatus into the patient by optionally using depth indicator markings on the exterior of the steerable catheter apparatus, for example on the exterior of the deflector. Additionally, alternatively or optionally, medical imaging is used to verify positioning. For example, the steerable catheter apparatus is provided with markers which will show to the attending medical professional using known medical imaging equipment.

In some embodiments of the invention, the steerable catheter apparatus is provided with a plurality of lumens, for example to deliver both eggs and semen and/or to evacuate and/or sample. In some embodiments of the invention, the deflector of the steerable catheter apparatus is used to also shield the cervix from movement of the steerable catheter apparatus, for example by providing a low friction and/or smooth and/or pliable exterior surface to the deflector. In some embodiments of the invention, the steerable catheter apparatus is connected to a syringe for injecting the eggs and/or semen into the patient.

In some embodiments of the invention, a guide wire is used to direct movement of the steerable catheter apparatus.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A steerable deflector, comprising:
a stylet configured with at least one groove, where the groove is a missing section disposed across only a part of a cross-section of the stylet; and,
a pull wire located on the outside of the stylet and configured to cause bending of the stylet at the at least one groove, said pull wire being attached together on the exterior of the stylet and extending distally of the groove by at least one of laser welding and an over-layer positioned around the deflector where the over-layer holds the pull wire in close proximity to the stylet when the pull wire is pulled to cause bending.

2. A steerable deflector according to claim 1, further comprising a handle connected to a proximal end of the stylet, where the handle is removably connected to a catheter.

3. A steerable deflector according to claim 1, further comprising a catheter, wherein the stylet is detachably located within a lumen of the catheter such that bending of the stylet by the pull wire also causes bending of the catheter.

4. A steerable deflector according to claim 1, further comprising a handle with a lever located on the handle and connected to the pull wire, wherein movement of the lever effectuates movement of the pull wire and bending of the stylet.

5-6. (canceled)

7. A steerable deflector according to claim 1, where the over-layer is heat shrunk on the deflector.

8. A steerable deflector according to claim 4, where the lever is provided with a plurality of removably lockable positions.

9. A steerable deflector according to claim 8, where the plurality of removably lockable positions correlate to known degree increments of bending of the deflector.

10. A steerable deflector according to claim 1, including a plurality of grooves.

11. A steerable deflector according to claim 10, where each of the plurality of grooves corresponds to a single pull wire for individual control of bending of each groove.

12. A steerable deflector according to claim 1, further comprising a catheter with multiple lumens where the stylet is detachably located in one of the lumens.

13. A steerable deflector according to claim 1, where the stylet and the pull wire together have a substantially circular cross-section.

14-16. (canceled)

17. A steerable deflector according to claim 3, further comprising a sharpened tip located at the distal end of the catheter for retrieving samples.

18. A steerable deflector according to claim 1, where the groove of the deflector is bendable up to 90 degrees from the longitudinal axis of the deflector.

19. A steerable deflector kit, comprising:
a plurality of catheters; and,
a steerable deflector according to claim 1, configured to be compatible with any of the plurality of catheters.

20. A steerable deflector kit according to claim 19, wherein at least one of the plurality of catheters is configured for performing a CVS, IVF or IUI procedure.

21. A method of using a steerable deflector, comprising:
inserting the steerable deflector including a stylet and a catheter;
navigating the steerable deflector to a target area, wherein navigating includes at least one of deflecting the deflector at a groove located thereon; and,
removing at least a portion of the steerable deflector.

22. (canceled)

23. A method according to claim 21, wherein removing includes removing the stylet but leaving the catheter in situ.

24. A method according to claim 23, further comprising attaching a syringe to the catheter for retrieving a sample from the target area.

25. A method according to claim 23, further comprising depositing at least one of semen and fertilized eggs at the target area.

26. A method according to claim 21, further comprising using a sharpened tip to retrieve a sample from the target area.

27. (canceled)
